(19) United States
(12) Patent Application Publication
(10) Pub. No.: US 2004/0087898 A1
(43) Pub. Date: May 6, 2004

(54) Breast pump assembly

(76) Inventor: Gotthilf Weniger, Trout Valley, IL (US)

Correspondence Address: Michael J. Turgeon, VEDDER PRICE KAUFMAN & KAMMHOLZ, 24TH FLOOR, 222 N. LASALLE STREET, CHICAGO, IL, 60601, US

(21) Appl. No.: 10/285,873

(22) Filed: Nov. 1, 2002

(51) Int. Cl. A61M 1/06

(52) U.S. Cl. 604/74

(57) ABSTRACT

A breast milk pump assembly for use in connection with electric or manual vacuum sources including a collapsible membrane for communicating a vacuum from the vacuum source to a breast shield.

### BACKGROUND OF THE INVENTION

The present invention relates to generally to a breast milk pump, and more particularly relates to an improved breast pump assembly having a vacuum isolation element, improved vacuum sources and ergonomic features.

Generally, breast pumps are well known and usually include a hood or shield that receives the human female breast, a vacuum source connected to the shield for generating and intermittent vacuum (or negative pressure) within the shield, and a receptacle container for the expressed milk. The intermittent suction action of the vacuum source serves to pull on the breast and nipple and thereby extract milk. The extracted milk typically flows from the shield into a collection container for storage and later use.

Inserts for use within the shield of the breast pump are known, but they have typically been used for sizing of the breast shield. Soft inserts in breast pumps are also known in the art, as disclosed by U.S. Pat. No. 4,799,922. A drawback of many soft inserts is that they tend to pinch the nipple as the insert contracts under vacuum causing discomfort and irritation. However, stroking or squeezing of the sides of the breast during lactation enhances the expression of milk. Such action is more like the suckling of an infant.

While various prior art breast pump constructions have been quite effective, there are certain drawbacks associated with some of these constructions. For example, there has been a problem with bacteriological contamination in some previous breast pumps when a motor driven pump is used. Overflow protection is necessary to prevent milk from entering the pump airline. Even so, it has been found that moisture and like from the user may still enter the pump airline.

Another design consideration for breast pumps is associated in the maintenance of the breast pump in a sanitary condition. In particular, depending upon the configuration of the interior walls of the hood body, it can be difficult for some users to properly clean the device. More specifically, the internal wall construction of the hood body of some previous constructions have crevices and small passage ways that are not easily cleaned. Further, other prior art breast constructions incorporate disposable inserts which must be removed and replaced after each use. While these constructions have simpler cleaning maintenance requirements, the constant replacement of the disposable parts increases the cost to the consumer considerably.

The vacuum sources in the form of electric pumps or hand pumps are known. However, rarely does a breast pump assembly provide sufficient structure for the user to select which desired method to use. Often, prior art designs favor one vacuum source over the other such that the unfavored source is usually not available to the user and if so, it is, at best, very difficult for the user to manipulate effectively. As a result, the prior art lacks a breast pump assembly fully capable of effective use with either vacuum source.

Further, use of a positive pressure source has been described in connection with expressing milk from a cow's udder, for example, U.S. Pat. Nos. 2,164,706, issued to Flint et al. and 3,659,558, issued to Noorlander. However, the construction or gross anatomy of the human female breast is significantly different from the cow's udder as discussed in The Mechanics of Different Methods of Emptying the Female Breast, E. Egnell, Journal of the Swedish Medical Association, vol. 40, 1956. Hence the novel construction and methods discussed herein overcome prior art disadvantages.

Therefore, there is a need in the art for a breast pump assembly which isolates the vacuum source from the expressed milk, is easy to use due to its design and provides true dual purpose functionality. Moreover, there exists a need in the art for a breast pump assembly including various aids for comforting the user and promoting the expression of milk.

### BRIEF DESCRIPTION OF THE DRAWINGS

In the course of the detail description, the reference will frequently be made to the attached drawings in which:

FIG. 1 is a perspective illustration of one embodiment of the new improved breast pump assembly constructed in accordance with the principles of the present invention;

FIG. 2 is a perspective view of one embodiment of the present invention in shown in FIG. 1;

FIG. 3 is an exported view of a breast pump assembly shown in FIG. 1;

FIG. 4 is a cross sectional view of the breast pump assembly as shown in FIG. 1;

FIG. 5 is a perspective view of another embodiment of the present invention showing a hand pump vacuum source;

FIG. 6 is an exploded view of the breast pump assembly as shown in FIG. 5;

FIG. 7 is a sectional view of the hand pump vacuum source of the breast pump assembly of FIG. 5;

FIG. 8 is another embodiment of the breast pump assembly of the present invention;

FIG. 9 is another embodiment for the breast pump assembly of the present invention;

FIG. 10 is another embodiment of the breast shield assembly of the present invention; and

FIG. 11 is a detailed course of view of the insert of FIG. 11 deformed in accordance with this embodiment of the present invention.

### DETAIL DESCRIPTION OF THE PREFERRED EMBODIMENTS

The present invention is directed to a breast pump assembly for expressing milk from the mammary glands of the human female. The breast pump assembly includes an interface connected to a breast shield, a container and a vacuum source. The interface includes a base portion and a mounting flange interconnected by a bracket. The base portion engages the container and includes a conduit therethrough to facilitate collection of the expressed milk in a container. The mounting flange includes a first side, a second side, a first pair of coupling elements and a port extending through the mounting flange. The first side includes a mounting boss for providing connection to the vacuum source, and the second side includes an outer edge. The breast shield includes a funnel portion, a vacuum chamber, a second pair of coupling elements and an outlet. The funnel portion includes a flared element and a tubular element. The vacuum chamber has a hemispherical configuration defining a recessed having a rim complimentary to the outer edge. The vacuum chamber is in communication with the funnel portion and the outlet to define a passageway. The outlet is in communication with the conduit. A hemispherical membrane defines a cavity complementary to the vacuum chamber and in communication with the vacuum source. The hemispherical membrane includes a shoulder formed about an open end thereof for engaging the outer edge of the mounting flange. The shoulder is disposed between the outer edge and the rim when the first and second coupling elements are engaged such the membrane collapses in response to a negative pressure generated by the vacuum source so that a vacuum is generated in the vacuum chamber to aid in expressing milk from the mammary glands, and the vacuum source is isolated from the expressed milk.

Another embodiment of the present invention is directed to a breast shield for use within a breast pump assembly including a vacuum source for expressing milk from the mammary glands of a human female. The breast shield includes a funnel portion which is adapted to receive a breast, an outlet, which is adapted to be in communication with the container, and a vacuum chamber. The vacuum chamber has hemispherical configuration and an open end defining a rim. The vacuum chamber is in communication with the funnel portion and the outlet to define a passageway for the expressed milk. A hemispherical membrane defines a cavity complimentary to the vacuum chamber which is adapted to be in communication with the vacuum source. The hemispherical membrane has a shoulder formed about an open end for engaging the rim and is disposed in the vacuum chamber such that the passageway is discontinuous. The hemispherical membrane generates a vacuum in the funnel portion by collapsing in response to a negative pressure on the membrane by the vacuum source in order to express milk and thereby establishes a continuous passageway for the expressed milk. The hemispherical membrane further isolates the passageway from the vacuum source.

Yet another embodiment of the present invention is directed to a breast shield assembly for use with a vacuum source including a funnel portion which is adapted to receive a breast. The funnel portion includes a heating element for warming the breast shield assembly prior to receiving the breast. A vacuum chamber is in communication with the funnel portion and an outlet to define a passageway. A membrane disposed within the vacuum chamber and complimentary shaped effective to communicate a vacuum from the vacuum source to within the funnel portion when in a collapsed state and to isolate the vacuum source from the expressed milk. The heating element enhances the comfort of the user and aids in the expression of the milk from the mammary glands.

Still another embodiment of the present invention is directed to a breast assembly for expressing milk from mammary glands from the human female which includes a pair of breast shield assemblies and a vacuum source. Each breast shield assembly includes a funnel portion adapted for receiving a breast therein and a collapsible hemispherical membrane including an interior cavity effective to communicating negative pressure from the vacuum source to within the funnel portion when disposed in a collapsed state and to the isolate the vacuum source from the breast. The vacuum source includes an electric pump having a pair pistons each in communication with a port and each independently in communication with one of the breast shield assemblies in order to provide a separate controllable vacuum to the collapsible membrane of each breast shield assembly.

Another embodiment of the present invention is directed to a breast pump assembly for expressing milk from a mammary gland from a human female which includes at least one breast shield assembly including a funnel portion adapted for receiving breast therein. A vacuum source for providing negative pressure within the funnel portion includes a device for generating musical sounds in response to activation of the vacuum source.

Yet another embodiment of the present invention is directed to a breast shield assembly for expressing milk from a mammary gland of the human female. The breast shield assembly includes a funnel portion including a flared element and a tubular element wherein the flared element is adapted to receive a breast. The funnel portion further includes inlet in communication with a source of positive pressure and a regulator for controlling the positive pressure. An insert disposed in complimentary configured to snuggly fit within the funnel portion is formed of flexible material and has a thinned portion disposed between the flared element and the tubular element. The thinned portion of the insert is deformable in response to the positive pressure in order to massage the breast and aid in the expression of milk from the mammary glands.

Still another embodiment of the present invention is directed to a breast pump assembly including an interface connected to a breast shield, a container and a vacuum source. The interface includes a mounted flange having a mounting boss formed on a first side thereof and a port extending through the mounting flange. The vacuum source includes an adapter and a handle. The adapter is operatively connected to the interface adjacent the mounting boss. The handle is connected to the adapter for relevant movement. The adapter further includes a base portion, an enlarged head, a column connecting the enlarged head to the base portion and uninterrupted length of compliant material disposed about an outer perimeter of the enlarged head. A bore is formed through the base portion, column and the enlarged head and is communication with the port. The handle includes an arm and enlarged portion disposed at a first end of the arm. The enlarged portion includes a base and sidewall which define a receptacle. The arm further includes a mount disposed between the first and second ends of the arm. The handle is operatively connected to the adapter at the mount for relative movement effective to generate a negative pressure in the bore and the port when the base is moved away from the enlarged head.

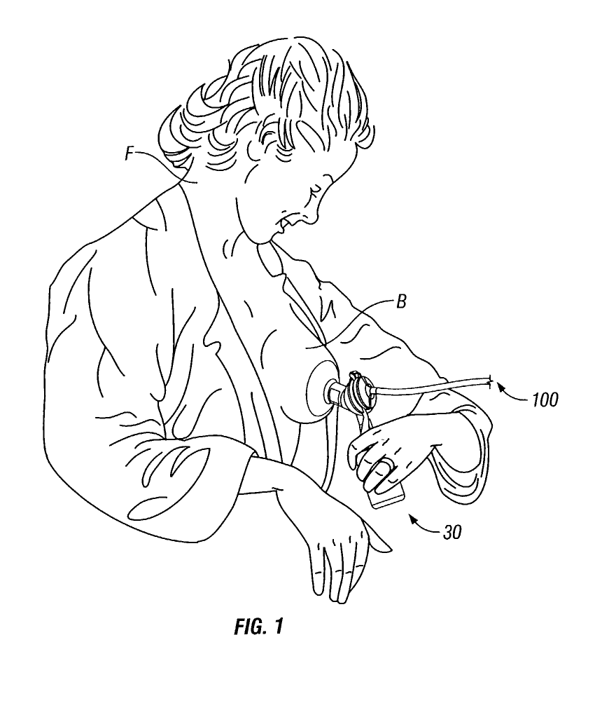

FIG. 1 is perspective view of one embodiment of the new and improved breast pump assembly for pumping and collecting expressed milk from the mammary glands from a human female constructed in accordance with the principles of the present invention. The breast pump assembly 30 is adapted to receive a breast B including the mammary glands of a human female F for expressing milk therefrom. A vacuum source is used in connection with the breast pump assembly for expressing the milk from the mammary glands in the breast B.

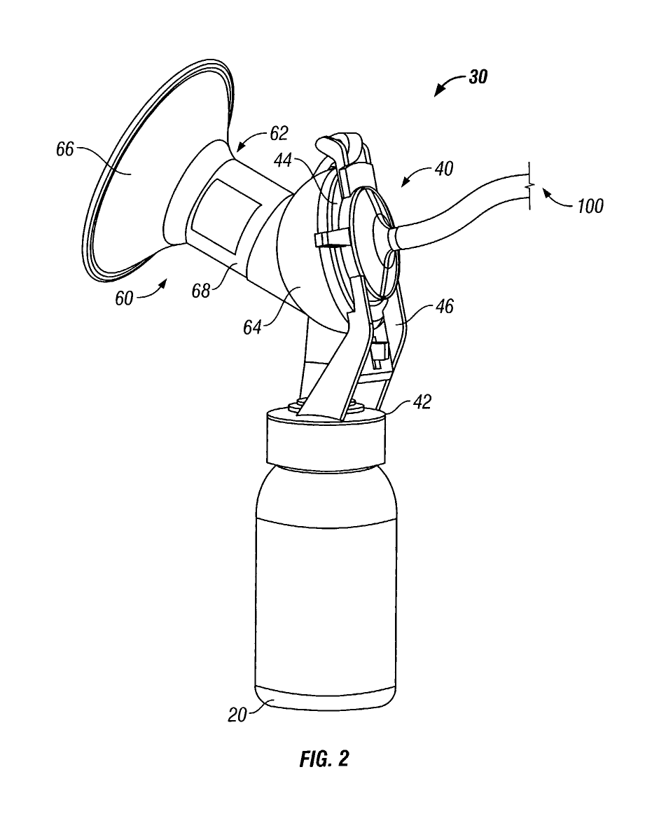

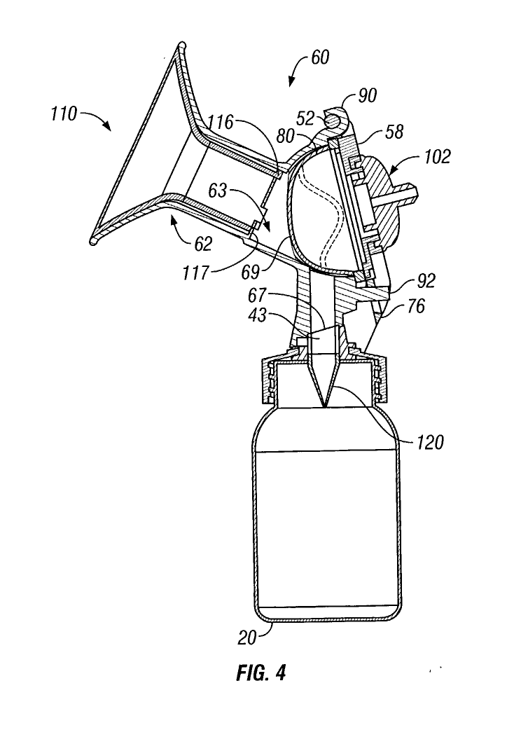

FIG. 2 is a perspective view of the embodiment of the breast pump assembly 30 as shown in FIG. 1 which includes an interface 40 connected to a breast shield 60 a container 20 and is used in connection with a vacuum source. The interface 40 includes a base portion 42 and a mounting flange 44 interconnected by a bracket 46. The base portion 42 engages the container 20 and includes a conduit (as shown in FIG. 4) therethrough to facilitate collection of the expressed milk in the container 20. The breast shield 60 includes a funnel portion 62, a vacuum chamber 64 and an outlet (as best shown in FIG. 4). The funnel portion 62 includes a flared element 66 and a tubular element 68. The vacuum chamber 64 has a hemispherical configuration defining a recess. The interface 40 and breast shield 60 are preferably formed of a molded, plastic construction. However, it is within the teachings of this invention that the interface 40 and breast shield 60 be formed by any suitable process of any suitable material. For example, the interface 40 and breast shield 60 may be formed by injection molding or other suitable process and the interface 40 and breast shield 60 are preferably constructed of polypropylene, polysulfone or polycarbonate or any other suitable material.

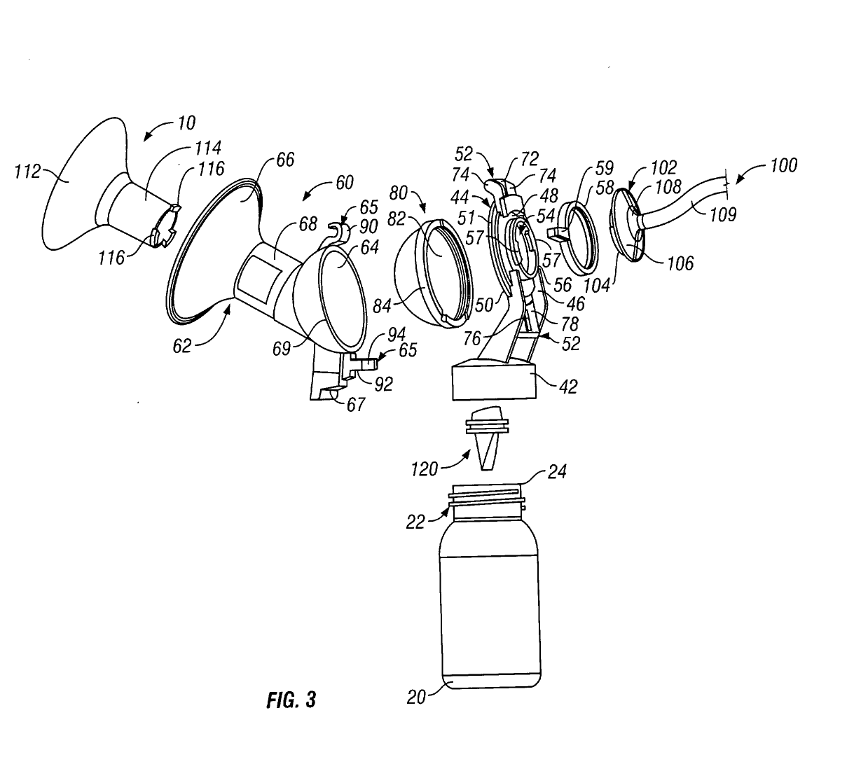

FIG. 3 is an exploded view of the breast pump assembly 30 in accordance with one embodiment of the present invention as shown in FIGS. 1 and 2. The breast pump 30 includes an interface 40 adapted to be connected to a breast shield 60, a container 20 and a vacuum source. The interface 40 includes a base portion 42 and a mounting flange 44 interconnected by a bracket 46. The base portion 42 includes structure by which the base portion 42 may engage the container 20. As shown, the container 20 includes a set of male screw-type threads 22 formed on a mouth portion 24 of the container 20. The base portion 42 includes a set of female screw threads (not shown) on an inner surface thereof for operative connection with the male threads 22 formed on the container 20. It is within the teaching of the present invention that other forms of operative connection such as push-and-turn connections, bayonet connections, press-fit connections or even snap-fit connections could be utilized for operatively connecting the base portion 42 to the container 20. It will be recognized by those of skill in the art that any other suitable operative connection which removably connects the base portion 42 to the container 20 may be used.

The mounting flange 44 includes a first side 48, a second side 50, and first pair of coupling elements 52 and a port 54 extending through the mounting flange 44. The first side 48 includes a mounting boss 56 for providing connection to the vacuum source. The mounting boss 56 further includes an opening to regulate the negative pressure from the vacuum source as is conventional in the prior art. It is within the teachings of the present invention that a regulator ring 58 may be connected to the mounting boss 56 such that movement of the regular ring 58 about the mounting boss 66 exposes an opening formed in the mounting boss 66 to atmosphere pressure to adjust or regulate the negative pressure from the vacuum source. The regulator ring 58 is connected to the exterior surface of the mounting boss 66 and further includes a handle 59 for moving the regulator ring 58 about the mounting boss 66 so as to the adjust or regulate the negative pressure from the vacuum source. The second side 50 includes an outer edge 51.

The breast shield 60 includes a funnel portion 62, a vacuum chamber 64, a second pair of coupling elements 65 and an outlet 67. The funnel portion 62 includes a flared element 66 and a tubular element 68. The vacuum chamber 64 has a hemispherical configuration which has a rim 69 complimentary to the outer edge 51. The vacuum chamber 64 is in communication with the funnel portion 62 and the outlet 67 to define a passageway (as best shown in FIG. 4). The breast shield 60 may be formed such that the funnel portion 62 is removably connected in order to accommodate various different breast sizes more accurately for increased comfort for the user.

A hemispherical membrane 80 defining a cavity 82 complimentary to the vacuum chamber 64 and in communication with the vacuum source includes a shoulder 84 formed about an open end thereof. The shoulder 84 engages the outer edge 51 of the mounting flange 44 such that the shoulder 84 is disposed between the outer edge 51 and the rim 69 when the first and second coupling elements 52, 65 are engaged. The membrane 80 collapses in response to a negative pressure generated by the vacuum source so that a vacuum is generated in the vacuum chamber 64 in order to express milk from the mammary glands. Further, the vacuum source is physically isolated from the expressed milk. The hemispherical membrane 80 may be removed when the first and second coupling elements 52, 65 are disengaged.

The first coupling elements 52 include a pivot bar 72 disposed at a distance from the mounting flange 44 by a pair of arms 74 connected to each of the pivot bar 72 and the mounting flange 44. The first coupling elements 52 further includes an aperture 76 formed in a latch plate 78 between the uprights of the bracket 46.

The second coupling elements 65 include a hook 90 formed on the an upper surface of the vacuum chamber 64 and a latch arm 92 disposed below the vacuum chamber 64 including an arrow-head type latch element 94. The hook 90 engages the pivot bar 72 such that the breast shield 60 is moveable about the pivot bar 72 between an open position and a closed position. When the breast shield 60 is moved from the open position to the closed position, the latch arm 92 and latch element 94 engage the aperture 76 in order to operatively connect the breast shield 60 to the interface 40.

The breast shield 60 may further include an insert 110 complimentary configured to the funnel portion 62 and receive therein for adapting the breast shield 60 for use with differently configured breasts. The insert 110 includes a flared element 112 and a tubular element 114. A plurality of lugs 116 engage a shoulder formed in the tubular element 68 of the breast shield 60 for positively securing the insert 110 to the breast shield 60.

The base portion 42 further includes a valve 120 in communication with the conduit (as best shown in FIG. 4) to control the flow of expressed milk into the container 20 and prevents the flow of expressed milk out of the container 20. The valve 120 is configured as a conventional duck-bill type valve which closes under vacuum and returns to an open position in the absence of vacuum. The valve 120 is in communication with the conduit such that milk is not trapped in the conduit, thereby reducing any possibility of bacteriological contamination. It is within the teachings of the present invention that other valves, or even other devices could be utilized for controlling the flow of expressed milk into the container and preventing the flow of expressed milk out of the container.

The mounting boss 66 further includes a pair of lugs 57 extending to the port 54 for connection with an adapter 102. The adapter 102 includes a mounting element 104, a top portion 106 and a nipple 108. The mounting elements 104 engage the lugs 57 in a conventional manner in order to sealingly secure the adapter 102 to the mounting boss 56. A hose 109 connects the adapter 102 to the vacuum source in order to supply vacuum or negative pressure to the membrane 80.

FIG. 4 is a cross-sectional view of the breast assembly of the embodiment shown in FIGS. 1-3 as assembled in an operative configuration. The insert 110 is installed within the funnel portion 62 of the breast shield 60. The lugs 116 engage the shoulder 117 to retain the insert 110 as shown. The regulator ring 58 and adapter 102 are operatively connected to the interface. The shoulder 84 of the hemispherical membrane 80 is disposed about the outer edge 51 of the second side 50. The hook 90 may then engage the pivot bar 52 so that the breast shield 60 may be operatively connected to the interface when the latch arm 92 engages the aperture 76 to retain the breast shield 60 in the operative configuration.

The shoulder 84 of the hemispherical membrane 80 is disposed between the rim 69 and the outer edge 51 effective to communicate a vacuum from the vacuum source to within the funnel portion 62 when in a collapsed state (as shown in phantom) and to isolate the vacuum source from the breast.

The vacuum chamber 64 is in communication with the funnel portion 62 and the outlet 67 to a define a passageway 63 for expressed milk. The passageway is discontinuous (as shown in FIG. 4) when the hemispherical membrane 80 is disposed in an unaltered configuration. When the hemispherical membrane generates a vacuum in the funnel portion 62 by collapsing in response to a negative pressure to applied to the membrane 80 by the vacuum source (as shown in phantom), not only is milk expressed, but the passageway is made continuous such that the expressed milk may flow through the outlet 67 which is in communication with the conduit 43 of the base portion 42. The valve 120 is also in communication with the conduit to control the flow of expressed milk as described in detail above.

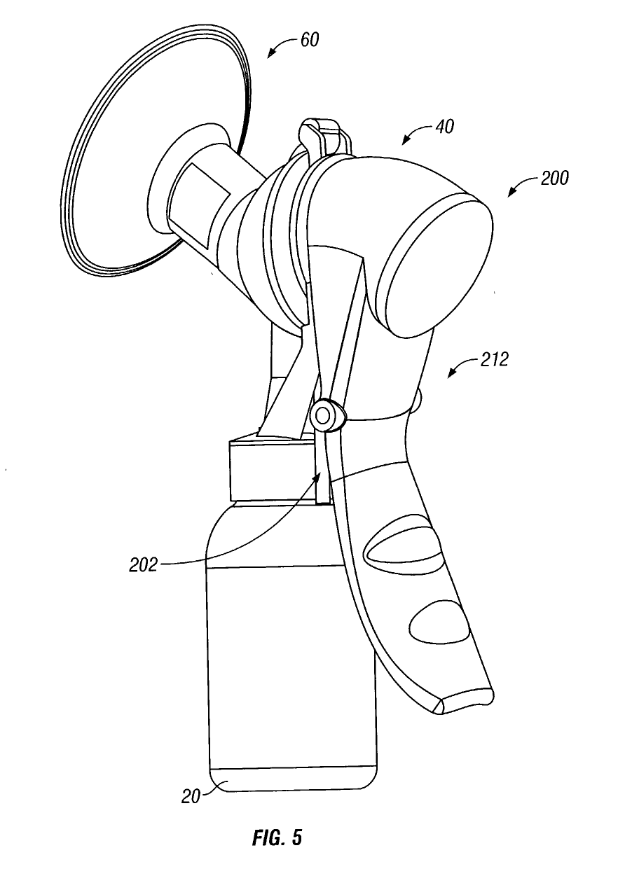

FIG. 5 is a perspective view of an alternative embodiment of the present invention. In this embodiment, the vacuum source includes a manually operated pump 200. Preferably, one hand of the user may manipulate this pump 200 in order to generate a negative pressure. As discussed above, an interface 40 is connected to a breast shield 60 and a container 20. An adapter 202 is operatively connected to the interface 40 adjacent the mounting boss. A handle 212 is connected to the adapter for relative movement. It will be recognized by those of skill in the art that the structure and function of the breast pump assembly other than the vacuum source will remain as described above.

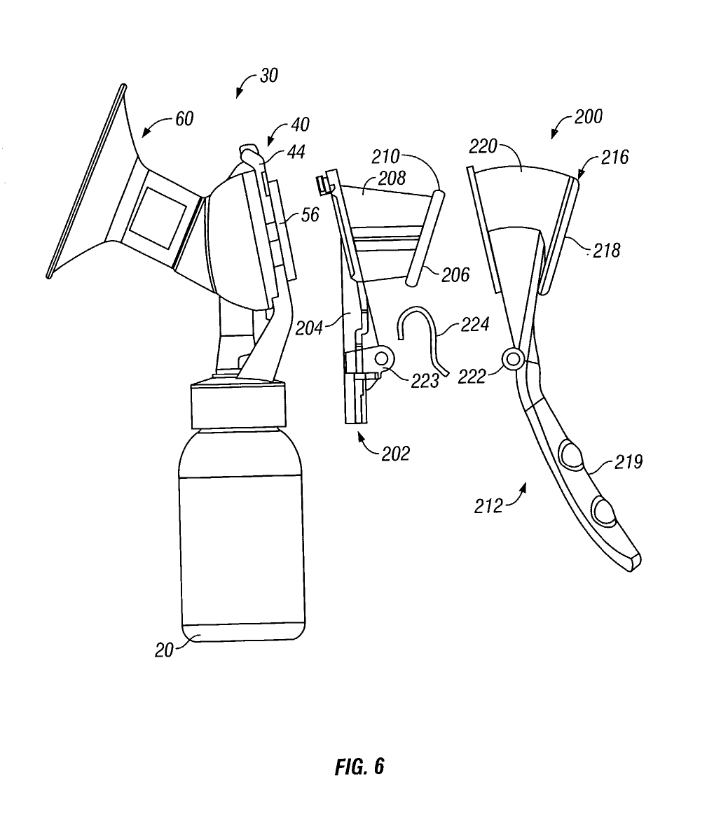

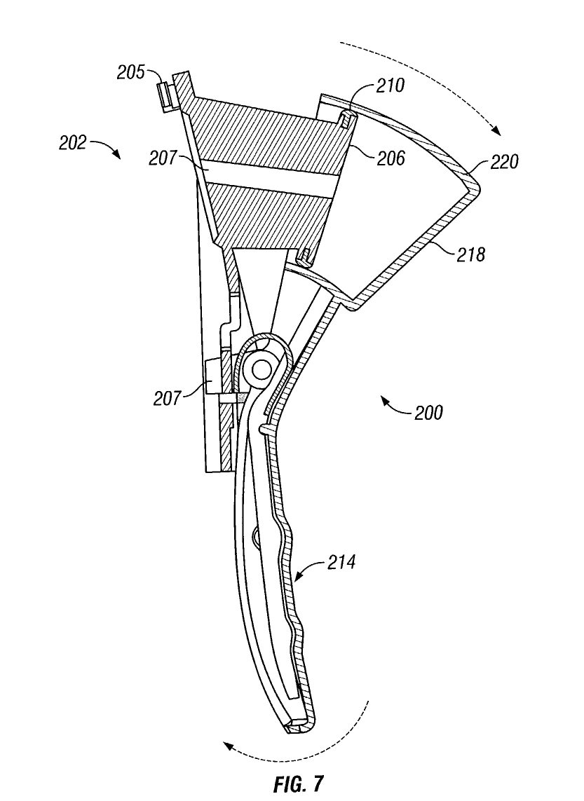

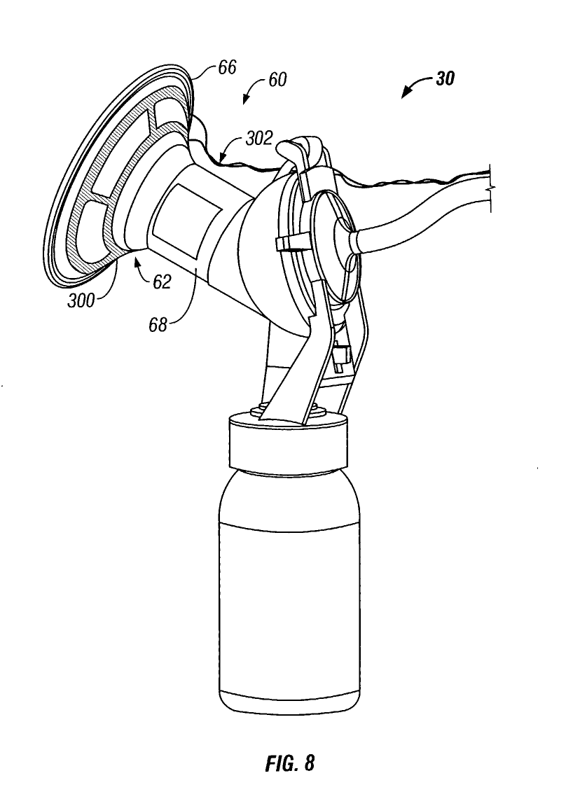

FIG. 6 is an exploded view of the embodiment of the present invention as shown in FIG. 5. The breast pump assembly 30 of this embodiment of the present invention includes an interface 40 connected to a breast shield 60, a container 20 and a vacuum source 200. The interface 40 includes a mounting flange 44 having a mounting boss 56 formed on a first side thereof and a port extending through the mounting flange. The vacuum source 200 includes an adapter 202 and a handle 212. The adapter 202 is operatively connected to the interface adjacent the mounting boss 56. The handle 212 is connected to the adopter 202 for relative movement. The adapter includes a base portion 204, an enlarged head 206, a column 208 connecting the enlarged head 206 to the base portion 204 and an uninterrupted length of compliant material 210 disposed about an outer perimeter of the enlarged head 206. A bore (as best shown in FIGS. 7 and 8) is formed through the base portion 204, column 208 and the enlarged head 206. The bore is in communication with the port of the interface 40.

The handle 212 includes an arm 214 and an enlarged portion 216 disposed at a first end of the arm 214. The enlarged portion 216 includes a base 218 and a side wall 220 to define a receptacle (as best shown in FIGS. 7 and 8). The arm 214 further includes a mount 222 disposed between the first end and the second end of the arm 214. The handle 214 is operatively connected to the adapter 202 by the mount 222 for relative movement effective to generate a negative pressure in the bore and the port when the base 218 is moved away from the enlarged head 206.

A pin or other suitable device engages the mount 222 and the fixture 223 to operatively connect the handle 214 to the adapter 202. A spring 224 may be disposed between the adapter 202 and handle 214 to normally bias the base 218 to a position adjacent the enlarged head 206. Accordingly, in operation, the arm 214 is moved against the biasing force of the spring 224 so that the base 218 is moved away from the enlarged head 206. The biasing force of the spring 224 then returns the arm 214 and the base 218 to their original positions and thereby a cycle is defined. The user continues to cycle the breast pump assembly in order to express the desired amount of milk from the mammary glands.

As discussed above, all other available structure and functionality of the breast pump assembly previously described are present in this embodiment of the present invention.

FIG. 7 is a cross-sectional view of the adapter 202 and handle 200 operatively connected for cycling between a first operative position as shown in FIG. 5 where the base 218 is disposed adjacent enlarged head 206 and a second operative position, as shown in FIG. 7, where the base 218 is moved away from the enlarged head 206. When the arm 214 is moved from the first operative position to the second operative position, a negative pressure is generated in the bore 207 which is then communicated through the port of the interface to the recess defined by the hemispherical membrane and collapses the membrane as discussed above in order to generate a vacuum or negative pressure in the vacuum chamber in order to facilitate the expression of milk.

The uninterrupted length of compliant material 210 is commonly referred to as an O-ring which maintains constant contact with the side wall 220 throughout the movement of the handle 200 from the first operative position to the second operative position. It will be recognized by those of skill in the art that the side wall 220 is configured such that the side wall 220 is always in circumferential contact with the O-ring 210 throughout the range of movement from the first operative position to the second operative position. The O-ring 210 is preferably formed from a resilient material and has a general mushroom or T-shaped cross-section. However, it is within the teachings of this invention that any suitable material or structural cross-section may provide the required functionality.

The adapter 202 further includes a latching element 205 which engages a portion of the interface adjacent the arms 74 of the first pair of coupling elements 52. A latch arm 207 engages the apertures 76 of the first pair of coupling elements 52 in order to positively secure the adapter 202 to the interface.

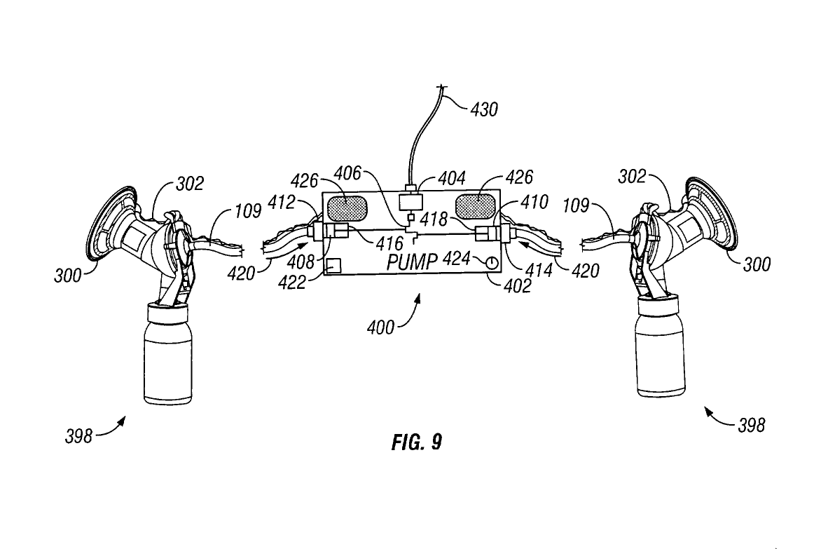

FIG. 8 is a perspective view of another embodiment of the breast pump assembly constructed in accordance with the principles of the present invention. The breast pump assembly 30, as shown in FIG. 8, is generally the same as discussed above with respect to FIGS. 1-4 in terms of structure and function. In this embodiment, a heating element 300 is provided on the flared element 66 of the funnel portion 62 of the breast shield assembly 60. Control leads 302 are connected to the heating element 300 and extends to a control device operatively associated with the electric vacuum pump as discussed in FIG. 9. The heating element 300 is preferably an electrically resistive element, or any other suitable device useful for warming the material of the breast shield 60. It will be recognized by those of skill in the art that the breast shield 60 may be formed from any suitable material of construction approved for use in contact with human skin. Preferably the breast shield 60 is formed from a plastic material and may be silicone-based.

It will be further recognized by those of skill in the art that the heating element 300 may be disposed on the flared element 66, tubular element 68 or a combination of the two. Furthermore, the heating element may be disposed on the funnel portion on the exterior surface thereof, embedded within the material or on the interior surface thereof. Preferably, for ease of construction, the heating element 300 is formed on the exterior surface of the funnel portion 62.

The heating element 300 is particularly useful for warming the breast shield prior to receiving the breast which enhances the comfort of the user. Furthermore, the warmed breast shield aids in the expression of milk from the mammary glands and provides therapeutic value. The increased temperature opens the milk ducts and enhances release of the milk in the engorged breast. It is within the teaching of this invention that the heating element 300 may be a device which increases the temperature of breast shield or any other suitable device which achieves the functionality discussed above.

In this embodiment, the breast shield may further include an insert complimentary configured to the funnel portion 62 and received therein for adapting the breast shield 60 for use with differently configured breasts. It will be recognized by those of skill in the art that the heating element 300 will also warm the insert to achieve the above described advantages.

FIG. 9 is a schematic representation of another embodiment of the breast pump assembly constructed in accordance with the present invention. In this embodiment, a pair of breast pumps assemblies 30, as described above with respect to FIGS. 1-4 or as shown in FIG. 8, are connected to a vacuum source configured as an electric pump 400. The pump 400 has a housing 402 for enclosing a motor 404 which is connected to a crank shaft 406 for reciprocating a pair of pistons 408, 410. Each piston is independently in communication with a port 412, 414 which is each independently in communication with one of the breast shield assemblies 398 in order to provide a separately controllable vacuum to the collapsible membrane of each breast shield assembly 398.

A vacuum is generated when the respective piston 408, 410 is withdrawn in the cylinder 416, 418 and accordingly pulls the air in the supply tube 109 and the collapsible membrane in the direction of arrow 420. This causes the collapsible membrane in each breast shield assembly 398 to collapse (as shown in phantom in FIG. 4).

Each breast shield assembly 398 has a regulator ring connected to a mounting boss such that movement of the regulator ring about the mounting boss exposes an opening formed in the mounting boss to atmospheric pressure to adjustably regulate the negative pressure from the vacuum source or pump 400. In this embodiment, the amount of negative pressure or vacuum experienced by the user is independently adjustable for each breast. This increases the comfort to the user and is particularly useful where each breast responds better to a different vacuum pressure. Further, separate adjustments at the breast shield allows the user to compensate for injured, tender or sore breasts.

Control leads 302 extend from the heating element 300 on each breast shield assembly 398 to the pump housing 402, wherein the control leads 302 are connected to a control device 422 which allows the user to adjust the intensity of the heating element 300. It will be recognized by those with skill in the art that the structure and function of the control device 422 will vary in accordance with the type of heating element 300 used. Where an electrically resistive heating element 300 is used, the control device 422 may be configured as a thermostat, or integrated circuit. Further, it will be recognized that other control devices of similar function which are suitable for such application may be substituted therefore.

In another embodiment of the present invention, the breast pump assembly for expressing milk from a mammary gland of human female, may include a device for generating musical sounds in response to activation of the vacuum source. In this embodiment, the pump housing 402 further includes a control knob 424 and speakers 426. When the vacuum source pump 400 is activated, a conventional device for generating musical sounds reproduces such musical sounds through the speakers 426. The control knob 425 allows the user to adjust the volume of the musical sounds. Preferably, the device for generating musical sounds is a computer memory storage device on which is saved data files representative of musical sounds generally recognized as comforting music. It will be recognized by those of skill in the art that any suitable other device for generating musical sounds may be used. For example, a compact disc player, a magnetic tape player, a MP3 player or other suitable device. Furthermore, headphones may also be used in place of the speakers. Electrical power is supplied to the vacuum source electric pump 400 by power cable 430. Electrical distribution within the pump housing 402 is accomplished in a conventional manner.

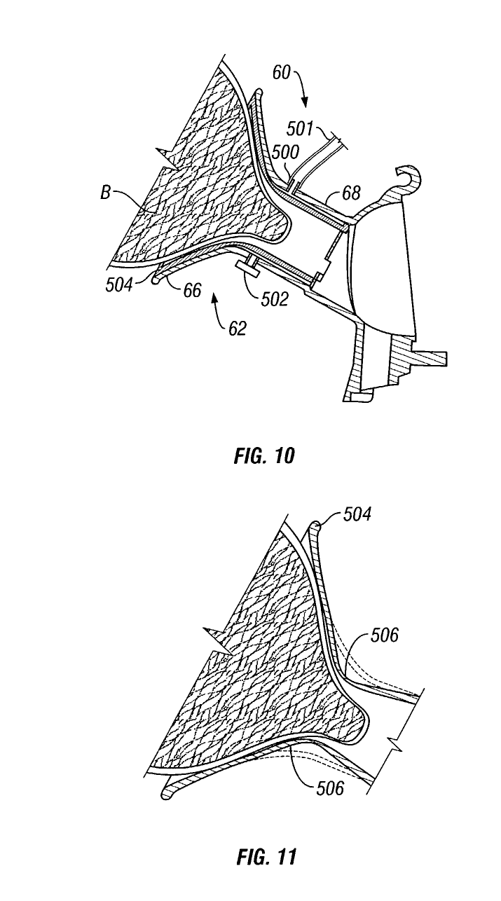

FIG. 10 is a cross-sectional view of another embodiment of the breast shield assembly 60 of the present invention. The funnel portion 62 includes a flared element 66 and a tubular element 68. The flared element 62 is adapted to receive a breast B. The funnel portion 66 further includes an inlet 500 in communication with a source of positive pressure by a tube 501 and a regulator 502 for controlling the amount of positive pressure. An insert 504 similar to that same element described above with respect to FIGS. 3 and 4, is disposed and complimentary configured to snuggly fit within the funnel portion 62 is formed of a flexible material and has a thinned portion 506 disposed between the flared element 66 and the tubular element 68. The thinned portion 506 of the insert 504 is deformable in response to the positive pressure in order to massage the breast B and aid in the expression of milk from the mammary glands.

FIG. 11 illustrates the massaging action applied to the breasts by the insert 504. The lines shown in phantom illustrate the original orientation of the insert prior to application of positive pressure. The insert 504 shown in solid illustrates the deformation of the thinned portion 506 of the insert 504 upon application of the positive pressure. The regulator 503 is used by the user to achieve a comfortable level of massaging wherein expression of milk is enhanced, but not at the expense of comfort to the user. The source of positive pressure may be an electric pump or a hand pump. Preferably, the source of positive pressure is synchronized with the vacuum source such that the massaging action is timely applied with respect to the vacuum pulse in order to maximize expression of the milk from the mammary glands. The advantage of this embodiment of the present invention is that deformation of the insert is controlled so that the nipple and breast are not pinched as in the prior art.

While the insert 504 is preferably formed on a flexible material, it will be recognized by those of skill in the art that any material approved for use in contact with human skin may be used. For example, a silicone-based material or other suitable material providing the above described functionality may be used.

The regulator 502 is preferably configured in a manner similar to the regulator on the interface, in that an aperture is provided through which the positive pressure may be adjustably vented to the atmosphere. Thereby, the massaging effect of the positive pressure pulses may be adjustably controlled. The remainder of the breast pump assembly in this embodiment operates as described above in any of the mentioned embodiments.

The embodiments described above are illustrative and not restrictive. A scope of the invention is indicated by the claims rather than the foregoing description. The invention has been described in all foreseeable embodiments. Accordingly, all changes which come within the scope of the claims are intended to be embraced therein.

What is claimed is:

1. A breast pump assembly for expressing milk from the mammary glands of a human female comprising:
an interface connected to a breast shield, a container and a vacuum source;
the interface including a base portion and a mounting flange interconnected by a bracket;
the base portion engaging the container and including a conduit therethrough to facilitate collection of the expressed milk in the container;
the mounting flange including a first side, a second side, a first pair of coupling elements and a port extending through the mounting flange;
the first side including a mounting boss for providing connection to the vacuum source;
the second side including an outer edge;
the breast shield including a funnel portion, a vacuum chamber, a second pair of coupling elements and an outlet;
the funnel portion including a flared element and a tubular element;
the vacuum chamber having a hemispherical configuration, defining a recess having a rim complementary to the outer edge, the vacuum chamber in communication with the funnel portion and the outlet to define a passageway;
the outlet in communication with the conduit; and
a hemispherical membrane defining a cavity complementary to the vacuum chamber and in communication with the vacuum source, the hemispherical membrane including a shoulder formed about an open end thereof for engaging the outer edge of the mounting flange, wherein the shoulder is disposed between the outer edge and the rim when the first and second coupling elements are engaged such that the membrane collapses in response to a negative pressure generated by the vacuum source so that a vacuum is generated in the vacuum chamber in order to aid in expressing milk from the mammary glands and the vacuum source is isolated from the expressed milk.

2. The breast pump assembly as recited in claim 1, wherein the mounting boss includes an opening to regulate the negative pressure from the vacuum source.

3. The breast pump assembly as recited in claim 1, further including a regulator ring connected to the mounting boss, wherein movement of the regulator ring about the mounting boss exposes an opening formed in the mounting boss exposes an opening formed in the mounting boss to atmospheric pressure to adjustably regulate the negative pressure from the vacuum source.

4. The breast pump assembly as recited in claim 1, wherein the second coupling elements include a hook, for engaging a pivot bar of the first coupling elements formed on the mounting flange, and a latch arm, for engaging a latch aperture of the first coupling elements formed in the interface.

5. The breast pump assembly as recited in claim 1, wherein the breast shield further includes an insert complimentary configured to the funnel portion and received therein for adapting the breast shield for use with differently configured human breasts.

6. The breast pump assembly as recited in claim 1, wherein the hemispherical membrane may be removed when the first and second coupling elements are disengaged.

7. The breast pump assembly as recited in claim 1, wherein the vacuum source includes an electric pump.

8. The breast pump assembly as recited in claim 1, wherein the vacuum source includes a hand-operated pump.

9. The breast pump assembly as recited in claim 1, wherein the base portion further includes a valve in communication with the conduit to control the flow of expressed milk into the container and prevents the flow of expressed milk out of the container.

10. A breast shield for use within a breast pump assembly including a vacuum source for expressing milk from the mammary glands of a human female, comprising:
a funnel portion in which a breast is adapted to be received, an outlet, which is adapted to be in communication with a container, and a vacuum chamber;
the vacuum chamber having a hemispherical configuration and an open end defining a rim, the vacuum chamber in communication with the funnel portion and the outlet to define a passageway for the expressed milk; and
a hemispherical membrane defining a cavity complementary to the vacuum chamber adapted to be in communication with the vacuum source, the hemispherical membrane having a shoulder formed about an open end for engaging the rim and disposed such that the passageway is discontinuous, wherein the hemispherical membrane generates a vacuum in the funnel portion by collapsing in response to a negative pressure applied to the membrane by the vacuum source in order to express milk and thereby establishes a continuous passageway for the expressed milk, and wherein the hemispherical membrane isolates the passageway from the vacuum source.

11. The breast shield as received in claim 10, wherein the breast shield further includes an insert complimentarily configured to the funnel portion and received therein for adapting the breast shield for use with differently configured human female mammary glands.

12. A breast shield assembly for use with a vacuum source, comprising:
a funnel portion in which a breast is adapted to be received including a heating element formed on the funnel portion for warming the breast shield assembly prior to receiving the breast such that after receiving the breast the milk ducts will open more easily and release of milk from the engorged breast is enhanced;
a vacuum chamber in communication with the funnel portion and an outlet to define a passageway; and
a container for collection of expressed milk.

13. The breast shield assembly as recited in claim 12, wherein the heating element includes a resistive element responsive to a remotely disposed control device.

14. The breast shield assembly as recited in claim 12, wherein the membrane is configured hemispherical.

15. The breast shield assembly as recited in claim 12, wherein the breast shield further includes an insert complimentarily configured to the funnel portion and received therein for adapting the breast shield for use with differently configured breasts.

16. The breast shield assembly as recited in claim 12, further including a membrane disposed within the vacuum chamber and complementary shaped effective to communicate a vacuum from the vacuum source to within the funnel portion when in a collapsed state and to isolate the vacuum source from the breast, wherein the heating element enhances the comfort of the human female and aids in the expression of milk from the mammary glands.

17. A breast pump assembly for expressing milk from mammary glands of a human female, comprising:
a pair of breast shield assemblies and a vacuum source;
each breast shield assembly including a funnel portion adapted for receiving a breast therein and a collapsible hemispherical membrane having an interior cavity effective to communicate a negative pressure from the vacuum source to within the funnel portion when disposed in a collapsed state and to isolate the vacuum source from the breast; and
the vacuum source including an electric pump having a pair of pistons, each in communication with a port and each independently in communication with one of the breast shield assemblies in order to provide a separately controllable vacuum to the collapsible membrane of each breast shield assembly.

18. The breast pump assembly as recited in claim 16, wherein the breast shield further includes an insert complimentarily configured to the funnel portion and received therein for adapting the breast shield for use with differently configured human female mammary glands.

19. A breast pump assembly for expressing milk from a mammary gland of a human female, comprising:
at least one breast shield assembly including a funnel portion adapted for receiving a breast therein;
a vacuum source for providing a negative pressure within the funnel portion including a device for generating musical sounds in response to activation of the vacuum source.

20. The breast pump assembly as recited in claim 18, wherein the vacuum source includes an electric pump having a pair of pistons, each piston in communication with a port and each piston is independently in communication with one breast shield assembly.

21. The breast pump assembly as recited in claim 18, wherein the device for generating musical sounds includes a reproduction device and a speaker.

22. The breast pump assembly as recited in claim 20, wherein the reproductive device is selected from the group consisting of an integrated chip, a magnetic tape player, or a compact disc player.

23. The breast pump assembly as recited in claim 18, wherein each at least one breast shield assembly further includes a vacuum chamber having a hemispherical configuration operatively associated with a membrane complementarily configured to the vacuum chamber and effective to communicate a vacuum from the vacuum source to within the funnel portion when in a collapsed state and to isolate the vacuum source from the breast.

24. A breast shield assembly for expressing milk from a mammary gland of a human female, comprising:
a funnel portion including a flared element and a tubular element, the flared element adapted to receive a breast;
the funnel portion further including an inlet in communication with a source of positive pressure and a regulator for controlling the positive pressure; and
an insert disposed and complementary configured to snugly fit within the funnel portion which is formed of a flexible material and has a thinned portion disposed between the flared element and the tubular element, wherein the thinned portion of the insert is deformable in response to the positive pressure in order to massage the breast and aid in the expression of milk from the mammary glands.

25. The breast shield assembly as recited in claim 23, wherein the breast shield is a vacuum chamber having a hemispherical configuration operatively associated with a membrane complementarily configured to the vacuum chamber and effective to communicate a vacuum from a vacuum source to within the insert when in a collapsed state and to isolate the vacuum source from the breast.

26. The breast shield as recited in claim 24, wherein the vacuum source is selected from the group consisting of an electric pump or a hand pump.

27. A breast pump assembly comprising:
an interface connected to a breast shield, a container and a vacuum source;
the interface including a mounting flange having a mounting boss formed on a first side thereof and a port extending through the mounting flange;
the vacuum source including an adapter and a handle;
the adapter operatively connected to the interface about the mounting boss;
the handle connected to the adapter for relative movement;
the adapter including a base portion, an enlarged head, a column connecting the enlarged head to the base portion and a uninterrupted length of compliant material disposed about an outer perimeter of the enlarged head;
a bore formed through the base portion, column and the enlarged head and in communication with the port;
the handle including an arm and enlarged portion disposed at a first end of the arm;
the enlarged portion including a base and a side wall defining a receptacle;
the arm further including a mount disposed between the first end and a second end of the arm;
the handle operatively connected to the adapter at the mount for relative movement effective to generate a negative pressure in the bore and the port when the base is moved away from the enlarged head.

28. The breast pump assembly as recited in claim 26, wherein the mounting boss includes an opening to regulate the negative pressure from the vacuum source.

29. The breast pump assembly as recited in claim 26, further including a regulator ring connected to the mounting boss, wherein movement of the regulator ring about the mounting boss exposes an opening formed in the mounting boss to atmospheric pressure to adjustably regulate the negative pressure from the vacuum source.

30. The breast pump assembly as recited in claim 24, wherein the breast shield further includes an insert complementarily configured to the funnel portion and received therein for adapting the breast shield for use with differently configured human female mammary glands.
